(19) United States
(12) Patent Application Publication
(10) Pub. No.: US 2018/0282257 A1
(43) Pub. Date: Oct. 4, 2018

(54) Aromatic Monoester Compositions and Processes for Preparing Same

(71) Applicant: ExxonMobil Chemical Patents Inc., Baytown, TX (US)

(72) Inventors: Abhimanyu O. Patil, Westfield, NJ (US); Satish Bodige, Wayne, NJ (US)

(21) Appl. No.: 15/913,985

(22) Filed: Mar. 7, 2018

Related U.S. Application Data
(60) Provisional application No. 62/477,665, filed on Mar. 28, 2017.

(51) Int. Cl. C07C 69/76; C07C 69/017; C07C 67/08; C10M 105/34; C10M 129/68; C10M 169/04

(52) U.S. Cl. CPC C07C 69/76; C07C 69/017; C07C 67/08; C10M 105/34; C10M 129/68; C10N 2230/74; C07C 2527/054; C07C 2531/025; C07C 2531/12; C10M 2207/2845; C10N 2230/02; C10M 169/042

(57) ABSTRACT

This disclosure relates to compositions that include one or more aromatic monoesters derived from branched Guerbet alcohols and aromatic acids, or from branched Guerbet acids and aromatic alcohols. The compositions are suitable as lubricant base stocks.

### CROSS-REFERENCE OF RELATED APPLICATIONS

This application claims the benefit of Provisional Application No. 62/477,665, filed Mar. 28, 2017, the disclosures of which is incorporated herein by reference.

### FIELD OF THE INVENTION

This disclosure relates to compositions that include one or more aromatic monoesters derived from branched Guerbet alcohols and aromatic acids or from branched to Guerbet acids and aromatic alcohols, processes for producing the compositions, lubricating oil base stocks, co-base stocks and lubricating oils containing the composition, and a method for improving one or more of solubility and dispersancy of polar additives of a lubricating oil by using as the lubricating oil a formulated oil containing the composition.

### BACKGROUND OF THE INVENTION

Lubricants in commercial use today are prepared from a variety of natural and synthetic base stocks admixed with various additive packages and solvents depending upon their intended application. The base stocks typically include mineral oils, polyalphaolefins (PAO), gas-to-liquid base oils (GTL), silicone oils, phosphate esters, diesters, polyol esters, and the like.

A major trend for passenger car engine oils (PCEOs) is an overall improvement in quality as higher quality base stocks become more readily available. Typically the highest quality PCEO products are formulated with base stocks such as PAOs or GTL stocks admixed with various additive packages.

Polyalpha-olefins (PAOs) are important lube base stocks with many excellent lubricant properties, including high viscosity index (VI), low volatility and are available in various viscosity range (e.g., kinematic viscosity at 100° C. in the range of 2 to 300 cSt). However, PAOs are paraffinic hydrocarbons with low polarity. This low polarity leads to low solubility and dispersancy for polar additives or sludge generated during service. To compensate for this low polarity, lube formulators usually add one or multiple polar co-base stocks. Ester or alkylated naphthalene (AN) is usually present at 1 to 50 wt % levels in many finished lubricant formulations to increase the fluid polarity which improves the solubility of polar additives and sludge.

Therefore, there is a need for oxygen containing polar Group V co-base fluids that provide appropriate solubility and dispersibility for polar additives or sludge generated during service of lubricating oils.

A major challenge in engine oil formulation is achieving improved appropriate solubility and dispersibility for polar additives or sludge generated during service of lubricating oils.

Therefore, there is need for better additive and base stock technology for lubricant compositions that will meet ever more stringent requirements of lubricant users. In particular, there is a need for advanced additive technology and synthetic base stocks with improved solubility and dispersibility for polar additives or sludge generated during service of lubricating oils.

The present disclosure also provides many additional advantages, which shall become apparent as described below.

### SUMMARY OF THE INVENTION

This disclosure provides compositions that include one or more aromatic monoesters derived from branched Guerbet alcohols and aromatic acids or from branched Guerbet acids and aromatic alcohols, processes for producing the compositions, and lubricating oil base stocks, co-base stocks and lubricating oils containing the composition. The compositions of this disclosure provide a solution for improving one or more of solubility and dispersancy of polar additives of a lubricating oil by using as the lubricating oil a formulated oil containing the composition.

A first aspect of the present invention relates to a composition comprising (i) a first compound having a formula (F-I) below, and/or (ii) a second compound having a formula (F-II) below:

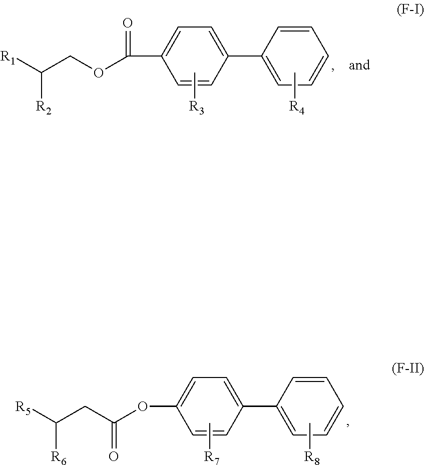

wherein R₁, R₂, R₅, and R₆ are independently a substituted or unsubstituted alkyl group having from 2 to 30 carbon atoms, and R₃, R₄, R₇, and R₈ are independently hydrogen or a substituted or unsubstituted alkyl group having from 1 to 8 carbon atoms.

The above composition can be advantageously a lubricant base stock to be incorporated into a lubricating oil.

A second aspect of the present invention relates to process for making a composition such as a lubricant base stock, comprising a step (A) or a step (B), or both steps (A) and (B), as follows: (A) reacting one or more Guerbet alcohols with one or more aromatic acids, optionally in the presence of a first catalyst and a first solvent, under reaction conditions sufficient to produce a first compound which comprises one or more Guerbet alcohol-based aromatic esters; wherein the Guerbet alcohol comprises a single branched mono-alcohol having from 8 to 32 carbon atoms, and the aromatic acid comprises an aromatic mono-carboxylic acid having from 8 to 32 carbon atoms; and (B) reacting one or more aromatic alcohols with one or more Guerbet acids, optionally in the presence of a second catalyst and a second solvent, under reaction conditions sufficient to produce a second compound which comprises one or more Guerbet acid-based aromatic esters; wherein the Guerbet acid comprises a single branched mono-carboxylic acid having from 8 to 32 carbon atoms, and the aromatic alcohol comprises an aromatic mono-alcohol having from 8 to 32 carbon atoms.

The composition prepared by the above process can be advantageously used as a base stock for incorporating into a lubricating oil.

Further objects, features and advantages of the present disclosure will be understood by reference to the following drawings and detailed description.

### DETAILED DESCRIPTION OF THE INVENTION

### Definitions

The term “Guerbet alcohol” refer to beta-substituted alcohol having a structure corresponding to the following structure (F-III):

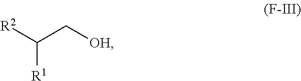

where R¹ and R² are independently linear, branched, cyclic, substituted or unsubstituted hydrocarbyl groups comprising from c1 to c2 carbon atoms, where c1 and c2 can be, independently, 1, 2, 3, 4, 5, 6, 7, 8, 9, 10, 15, 20, 25, 30, 35, 40, 45, 50, 55, 60, 65, 70, 75, 80, 85, 90, 95, 100, as long as c1<c2. Preferably c1≥2 and c2≤50. Preferably R¹ and R² are alkyls. More preferably R¹ and R² are linear or branched alkyls.

The term “Guerbet acid” refers to beta-substituted alcohol having a structure corresponding to the following structure (F-IV):

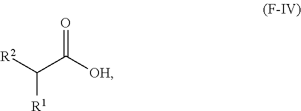

where R¹ and R² are independently linear, branched, cyclic, substituted or unsubstituted hydrocarbyl groups comprising from c1 to c2 carbon atoms, where c1 and c2 can be, independently, 1, 2, 3, 4, 5, 6, 7, 8, 9, 10, 15, 20, 25, 30, 35, 40, 45, 50, 55, 60, 65, 70, 75, 80, 85, 90, 95, 100, as long as c1<c2. Preferably c1≥2 and c2≤50. Preferably R¹ and R² are alkyls. More preferably R¹ and R² are linear or branched alkyls.

The term “monoester” refer to ester having one ester (—C(O)—O—) functional group therein.

The term “aromatic acid” refer to an organic acid comprising a carboxylic group (—C(O)—OH) connected directly to an aromatic structure therein.

The term “aromatic alcohol” refers to an alcohol comprising a hydroxyl group (—OH) connected directly to an aromatic structure therein.

The term “alkyl” refers to a saturated hydrocarbyl group consisting of carbon and hydrogen atoms.

The term “lubricant” or “lubricating oil,” used interchangeably herein, refers to a substance that can be introduced between two or more surfaces and lowers the level of friction between two adjacent surfaces moving relative to each other. A lubricant “base stock” is a material, typically a fluid at the operating temperature of the lubricant, used to formulate a lubricant by admixing with other components. Non-limiting examples of base stocks suitable in lubricants include API Group I, Group II, Group III, Group IV, and Group V base stocks. PAOs, particularly hydrogenated PAOs, have recently found wide use in lubricant formulations as a Group IV base stock, and are particularly preferred. A lubricant can comprise one base stock, with or without additional base stocks. If one base stock is designated as a primary base stock in the lubricant, additional base stocks may be called a co-base stock.

All numerical values within the detailed description and the claims herein are modified by “about” or “approximately” the indicated value, and take into account experimental error and variations that would be expected by a person having ordinary skill in the art.

All kinematic viscosity values in the present disclosure are as determined according to ASTM D445. Kinematic viscosity at 100° C. is reported herein as KV100, and kinematic viscosity at 40° C. is reported herein as KV40.

All viscosity index (“VI”) values in the present disclosure are as determined according to ASTM D2270.

All Noack volatility (“NV”) values in the present disclosure are as determined according to ASTM D5800, unless otherwise specified.

All pour points values in the present disclosure are as determined according to ASTM D97.

### Compositions of the Present Disclosure

The compositions of this disclosure contain one or more aromatic monoesters derived from branched Guerbet alcohols and aromatic acids, or from branched Guerbet acids and aromatic alcohols. These compositions exhibit good solvency for polar additives, which make them attractive as Group V synthetic base stocks in lubricant applications. The compositions of this disclosure can be used as a primary base stock or a co-base stock in lubricants.

The compositions of this disclosure are oxygen-containing Group V base stocks. Unlike alkylated naphthalene Group V base stocks, the base stocks of this disclosure contain oxygen functionality and are more polar.

In accordance with this disclosure, the residue of a branched Guerbet alcohol is attached to an aromatic acid, or the residue of a branched Guerbet acid is attached to an aromatic alcohol, to obtain Group V fluids. By changing the Guerbet alcohol or Guerbet acid residue portion, molecules with varying polarity (and hydrocarbon compatibility) can be synthesized. These molecules can be used as base stocks or as co-base stocks along with mPAO (metallocene catalyst based poly-alpha-olefin), PAO, Group I-III+, GTL, ionic liquids, and the like.

As indicated above, aromatic monoester compound base stock and co-base stock components useful in this disclosure include, for example, compositions containing one or more compounds derived from a branched Guerbet alcohol and an aromatic acid (the first compound), represented by the formula

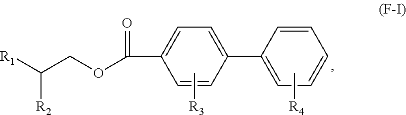

wherein R₁ and R₂ are independently a substituted or unsubstituted alkyl group having from 2 to 30 carbon atoms, and R₃ and R₄ are independently hydrogen or a substituted or unsubstituted alkyl group having from 1 to 8 carbon atoms.

Preferred aromatic monoester compound base stock and co-base stock components derived from a branched Guerbet alcohol and an aromatic acid include, for example, 2-hexyldecyl 4′-methylbiphenyl-4-carboxylate, 2-butyloctyl 4′-methylbiphenyl-4-carboxylate, 2-ethylhexyl 4′-methyl-[1,1′-biphenyl]-4-carboxylate, 2-octyldodecyl 4′-methyl-[1,1′-biphenyl]-4-carboxylate, 2-decyltetradecyl 4′-methyl-[1,1′-biphenyl]-4-carboxylate, 2-butyloctyl [1,1′-biphenyl]-4-carboxylate, 2-ethylhexyl [1,1′-biphenyl]-4-carboxylate, 2-hexyldecyl [1,1′-biphenyl]-4-carboxylate, 2-octyldodecyl [1,1′-biphenyl]-4-carboxylate, 2-decyltetradecyl [1,1′-biphenyl]-4-carboxylate, and mixtures thereof.

Aromatic monoester compound base stock and co-base stock components derived from a branched Guerbet alcohol and an aromatic acid have a KV100 from 3 to 12 cSt, a VI from 25 to 125, and a Noack volatility of less than 20% as determined by ASTM D5800.

Preferred aromatic monoester compound base stock and co-base stock components derived from a branched Guerbet alcohol and an aromatic acid have a KV100 from 4 to 11, more preferably from 5 to 10, a VI from 30 to 120, more preferably from 35 to 115, even more preferably from 40 to 110, and a NV of no greater than 18%, more preferably no greater than 15%, even more preferably no greater than 12%.

Also, as indicated above, aromatic monoester compound base stock and co-base stock components useful in this disclosure include, for example, compositions containing one or more compounds derived from a branched Guerbet acid and an aromatic alcohol (the second compound), represented by the formula

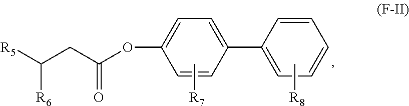

wherein R₅ and R₆ are the same or different and are a substituted or unsubstituted alkyl group having from 2 to 30 carbon atoms, and R₇ and R₈ are the same or different and are hydrogen or a substituted or unsubstituted alkyl group having from 1 to 8 carbon atoms.

Preferred aromatic monoester compound base stock and co-base stock components derived from a branched Guerbet acid and an aromatic alcohol include, for example, [1,1′-biphenyl]-4-yl 2-decyltetradecanoate, [1,1′-biphenyl]-4-yl 2-octyldodecanoate, [1,1′-biphenyl]-4-yl 2-hexyldecanoate, [1,1′-biphenyl]-4-yl 2-butyloctanoate, [1,1′-biphenyl]-4-yl 2-ethylhexanoate, [1,1′-biphenyl]-3-yl 2-decyltetradecanoate, [1,1′-biphenyl]-3-yl 2-octyldodecanoate, [1,1′-biphenyl]-3-yl 2-hexyldecanoate, [1,1′-biphenyl]-3-yl 2-butyloctanoate, [1,1′-biphenyl]-3-yl 2-ethylhexanoate, [1,1′-biphenyl]-2-yl 2-decyltetradecanoate, [1,1′-biphenyl]-2-yl 2-octyldodecanoate, [1,1′-biphenyl]-2-yl 2-hexyldecanoate, [1,1′-biphenyl]-2-yl 2-butyloctanoate, [1,1′-biphenyl]-2-yl 2-ethylhexanoate, 3′-methoxy-[1,1′-biphenyl]-2-yl 2-decyltetradecanoate, 3′-methoxy-[1,1′-biphenyl]-2-yl 2-octyldodecanoate, 3′-methoxy-[1,1′-biphenyl]-2-yl 2-hexyldecanoate, 3′-methoxy-[1,1′-biphenyl]-2-yl 2-butyloctanoate, 3′-methoxy-[1,1′-biphenyl]-2-yl 2-ethylhexanoate, 3-amino-[1,1′-biphenyl]-4-yl 2-decyltetradecanoate, 3-amino-[1,1′-biphenyl]-4-yl 2-octyldodecanoate, 3-amino-[1,1′-biphenyl]-4-yl 2-hexyldecanoate, 3-amino-[1,1′-biphenyl]-4-yl 2-butyloctanoate, 3-amino-[1,1′-biphenyl]-4-yl 2-ethylhexanoate, 3′-formyl-[1,1′-biphenyl]-4-yl 2-decyltetradecanoate, 3′-formyl-[1,1′-biphenyl]-4-yl 2-octyldodecanoate, 3′-formyl-[1,1′-biphenyl]-4-yl 1 2-hexyldecanoate, 3′-formyl-[1,1′-biphenyl]-4-yl 2-butyloctanoate, 3′-formyl-[1,1′-biphenyl]-4-yl 2-ethylhexanoate, and mixtures thereof.

Aromatic monoester compound base stock and co-base stock components derived from a branched Guerbet acid and an aromatic alcohol have a KV100 from 3 to 12 cSt, a VI of from 25 to 125, and a NV of less than 20%.

Preferred aromatic monoester compound base stock and co-base stock components derived from a branched Guerbet acid and an aromatic alcohol have a KV100 from 4 to 11, more preferably from 5 to 10, at 100° C. as determined by ASTM D-445, a viscosity index (VI) from 30 to 120, more preferably from 35 to 115, even more preferably from 40 to 110, as determined by ASTM D-2270, and a Noack volatility of no greater than 18%, more preferably no greater than 15%, even more preferably no greater than 12%, as determined by ASTM D-5800.

In accordance with this disclosure, a process is provided for producing a composition containing one or more Guerbet alcohol-based aromatic esters. The process involves reacting one or more Guerbet alcohols with one or more aromatic acids, optionally in the presence of a catalyst and a solvent, under reaction conditions sufficient to produce one or more Guerbet alcohol-based aromatic esters. The Guerbet alcohol is desirably a single branched mono-alcohol having from 8 to 32 carbon atoms, and the aromatic acid is desirably an aromatic mono-carboxylic acid having from 8 to 32 carbon atoms.

Illustrative Guerbet alcohols useful in the process of this disclosure include, for example, 2-hexyl-1-decanol, 2-butyloctanol, 2-hexyl-1-octanol, 2-hexyl-1-decanol, 2-octyl-1-decanol, 2-octyl-1-dodecanol, 2-decyl-1-dodecanol, 2-decyl-1-tetradecanol, 2-heptyl-1-undecanol, 2-ethyl-1-hexanol, 2-butyl-1-hexanol, 2-butyl-1-octanol, and mixtures thereof.

Illustrative aromatic acids useful in the process of this disclosure include, for example, 4′-methylbiphenyl-4-carboxylic acid, biphenyl-2-carboxylic acid, biphenyl-4-carboxylic acid, biphenyl-3-carboxylic acid, 4′-(trifluoromethyl)-2-biphenylcarboxylic acid, 4′-hydroxy-4-biphenylcarboxylic acid, 4′-methyl-4-biphenylcarboxylic acid, 4′-formyl-4-biphenylcarboxylic acid, 4′-bromo-4-biphenylcarboxylic acid, 3′-bromo-3-biphenylcarboxylic acid, 3′-(fluorophenyl)benzoic acid, 4′-amino-4-biphenylcarboxylic acid, 3′-amino-3-biphenylcarboxylic acid, 2-biphenyl-[1,3]dioxol-5-yl-carboxylic acid, 3-biphenyl-[1,3]dioxol-5-yl-carboxylic acid, 2-biphenyl-(2′-methoxy)acetic acid, 4-biphenyl[1,3]dioxo-5-yl-acetic acid, 4-biphenyl-3′-amino acetic acid, 5-amino-biphenyl-2-carboxylic acid, (2-methoxy-biphenyl-4-yl)acetic acid, 2-cyano-biphenyl-2-carboxylic acid, 4-cyano-biphenyl-2-carboxylic acid, 4-formyl-biphenyl-4-carboxylic acid, biphenyl-4-carboxylic acid, biphenyl-3-carboxylic acid, 1-naphthalenecarboxylic acid, 2-naphthalenecarboxylic acid, 4-methyl-l-napthoic acid, 1-napthoic acid, 2-napthoic acid, and mixtures thereof.

Illustrative catalysts useful in the process of this disclosure include, for example, p-toluenesulfonic acid monohydride (PTSA), titanium (VI) isopropoxide, or sulfuric acid.

Illustrative solvents useful in the process of this disclosure include, for example, toluene, xylene, hexane, or mixtures thereof.

Reaction conditions for the reaction of the one or more Guerbet alcohols with one or more aromatic acids, such as temperature, pressure and contact time, may also vary greatly and any suitable combination of such conditions may be employed herein. The reaction temperature may range from 25° C. to 250° C., and preferably from 30° C. to 200° C., and more preferably from 60° C. to 150° C. Normally the reaction is carried out under ambient pressure and the contact time may vary from a matter of seconds or minutes to a few hours or greater. The reactants can be added to the reaction mixture or combined in any order. The stir time employed can range from 0.5 to 48 hours, preferably from 1 to 36 hours, and more preferably from 2 to 24 hours.

The composition of the present invention may be a mixture or combination of a compound having formula (F-I) above and a compound having formula (F-II) above, at any proportion. Given the similarity of the molecular structures of (F-I) and (F-II), it is contemplated that a mixture of both types may behave quite similar to a pure base stock of (F-I) or (F-II).

Also, in accordance with this disclosure, a process is provided for producing a composition containing one or more Guerbet acid-based aromatic esters. The process involves reacting one or more aromatic alcohols with one or more Guerbet acids, optionally in the presence of a catalyst and a solvent, under reaction conditions sufficient to produce one or more Guerbet acid-based aromatic esters. The Guerbet acid is a single branched mono-carboxylic acid having from 8 to 32 carbon atoms, and the aromatic alcohol is an aromatic mono-alcohol having from 8 to 32 carbon atoms.

Illustrative Guerbet acids useful in the process of this disclosure include, for example, 2-ethylhexanoic acid, 2-butylhexanoic acid, 2-butyloctanoic acid, 2-hexyldecanoic acid, 2-heptylundecanoic acid, 2-octyldecanoic acid, 2-decyldodecanoic acid, isotridecanoic acid, and mixtures thereof.

Illustrative aromatic alcohols useful in the process of this disclosure include, for example, 1-naphthol, 2-naphthol, 2-methyl-1-naphthol, 3-methoxy-2-naphthol, 3-methoxynaphthalen-1-ol, 4-methoxy-1-naphthol, 6-methoxy-2-naphthol, 2-phenylphenol, 3-phenylphenol, 4-phenylphenol, 4-phenoxyphenol, 2-amino-4-phenylphenol, 3′-hydroxy-biphenyl-3-carbaldehyde, 4′-hydroxy-biphenyl-3-carbaldehyde, 3-methoxy [1,1′-biphenyl]-3-ol, and mixtures thereof.

Illustrative catalysts useful in the process of this disclosure include, for example, p-toluenesulfonic acid monohydride (PTSA), titanium (VI) isopropoxide or sulfuric acid.

Illustrative solvents useful in the process of this disclosure include, for example, toluene, xylene, or mixtures thereof.

Reaction conditions for the reaction of the one or more Guerbet acids with one or more aromatic alcohols, such as temperature, pressure and contact time, may also vary greatly and any suitable combination of such conditions may be employed herein. The reaction temperature may range from 25° C. to 250° C., and preferably from 30° C. to 200° C., and more preferably from 60° C. to 150° C. Normally the reaction is carried out under ambient pressure and the contact time may vary from a matter of seconds or minutes to a few hours or greater. The reactants can be added to the reaction mixture or combined in any order. The stir time employed can range from 0.5 to 48 hours, preferably from 1 to 36 hours, and more preferably from 2 to 24 hours.

Good high-temperature thermal and oxidative stability, good solvency for polar additives, and traction benefits, can be attained in an engine lubricated with a lubricating oil by using as the lubricating oil a formulated oil in accordance with this disclosure. In particular, a lubricating oil base stock comprising one or more aromatic monoester compounds exhibits desired solvency for polar additives, and oxidative and thermal stability, which helps to prolong the useful life of lubricants and significantly improve the durability and resistance of lubricants when exposed to high temperatures. The lubricating oils of this disclosure are particularly advantageous as passenger vehicle engine oil (PVEO) products.

Examples of techniques that can be employed to characterize the compositions formed by the process described above include, but are not limited to, analytical gas chromatography, nuclear magnetic resonance, thermogravimetric analysis (TGA), inductively coupled plasma mass spectrometry, differential scanning calorimetry (DSC), volatility and viscosity measurements.

This disclosure provides lubricating oils useful as engine oils and in other applications. The lubricating oils are based on high quality base stocks including a major portion of a hydrocarbon base fluid such as a PAO or GTL with a secondary co-base stock component which is an aromatic monoester compound as described herein. The lubricating oil base stock can be any oil boiling in the lube oil boiling range, typically from 100 to 450° C. In the present specification and claims, the terms base oil(s) and base stock(s) are used interchangeably.

The compositions of this disclosure are useful in other application, for example, PVC plasticizers.

The viscosity-temperature relationship of a lubricating oil is one of the critical criteria which must be considered when selecting a lubricant for a particular application. Viscosity Index (VI) is an empirical, unitless number which indicates the rate of change in the viscosity of an oil within a given temperature range. Fluids exhibiting a relatively large change in viscosity with temperature are said to have a low viscosity index. A low VI oil, for example, will thin out at elevated temperatures faster than a high VI oil. Usually, the high VI oil is more desirable because it has higher viscosity at higher temperature, which translates into better or thicker lubrication film and better protection of the contacting machine elements.

In another aspect, as the oil operating temperature decreases, the viscosity of a high VI oil will not increase as much as the viscosity of a low VI oil. This is advantageous because the excessive high viscosity of the low VI oil will decrease the efficiency of the operating machine. Thus high VI (HVI) oil has performance advantages in both high and low temperature operation.

### Lubricating Oil Base Stocks

A wide range of lubricating oils is known in the art. Lubricating oils that are useful in the present disclosure are both natural oils and synthetic oils. Natural and synthetic oils (or mixtures thereof) can be used unrefined, refined, or rerefined (the latter is also known as reclaimed or reprocessed oil). Unrefined oils are those obtained directly from a natural or synthetic source and used without added purification. These include shale oil obtained directly from retorting operations, petroleum oil obtained directly from primary distillation, and ester oil obtained directly from an esterification process. Refined oils are similar to the oils discussed for unrefined oils except refined oils are subjected to one or more purification steps to improve the at least one lubricating oil property. One skilled in the art is familiar with many purification processes. These processes include solvent extraction, secondary distillation, acid extraction, base extraction, filtration, and percolation. Rerefined oils are obtained by processes analogous to refined oils but using an oil that has been previously used as a feed stock.

Groups I, II, III, IV and V are broad categories of base oil stocks developed and defined by the American Petroleum Institute (API Publication 1509; www.API.org) to create guidelines for lubricant base oils. Group I base stocks generally have a viscosity index of from 80 to 120 and contain greater than 0.03% sulfur and less than 90% saturates. Group II base stocks generally have a viscosity index of from 80 to 120, and contain less than or equal 25 to 0.03% sulfur and greater than or equal to 90% saturates. Group III stock generally has a viscosity index greater than 120 and contains less than or equal to 0.03% sulfur and greater than 90% saturates. Group IV includes polyalphaolefins (PAO). Group V base stocks include base stocks not included in Groups I-IV. The table below summarizes properties of each of these five groups.

Base Oil Properties

| | Saturates | Sulfur | Viscosity Index |
|---|---|---|---|
| Group I | <90 and/or | >0.03% and | ≥80 and <120 |
| Group II | ≥90 and | ≤0.03% and | ≥80 and <120 |
| Group III | ≥90 and | ≤0.03% and | ≥120 |

| | |
|---|---|
| Group IV | Includes polyalphaolefins (PAO) products |
| Group V | All other base oil stocks not included in Groups I, II, III or IV |

Natural oils include animal oils, vegetable oils (castor oil and lard oil, for example), and mineral oils. Animal and vegetable oils possessing favorable thermal oxidative stability can be used. Of the natural oils, mineral oils are preferred. Mineral oils vary widely as to their crude source, for example, as to whether they are paraffinic, naphthenic, or mixed paraffinic-naphthenic. Oils derived from coal or shale are also useful in the present disclosure. Natural oils vary also as to the method used for their production and purification, for example, their distillation range and whether they are straight run or cracked, hydrorefined, or solvent extracted.

Group II and/or Group III hydroprocessed or hydrocracked base stocks, as well as synthetic oils such as polyalphaolefins, alkyl aromatics and synthetic esters, i.e. Group IV and Group V oils are also well known base stock oils.

Synthetic oils include hydrocarbon oil such as polymerized and interpolymerized olefins (polybutylenes, polypropylenes, propylene isobutylene copolymers, ethylene-olefin copolymers, and ethylene-alphaolefin copolymers, for example). Polyalphaolefin (PAO) oil base stocks, the Group IV API base stocks, are a commonly used synthetic hydrocarbon oil. By way of example, PAOs derived from C₈, C₁₀, C₁₂, C₁₄ olefins or mixtures thereof may be utilized. See U.S. Pat. Nos. 4,956,122; 4,827,064; and 4,827,073, which are incorporated herein by reference in their entirety. Group IV oils, that is, the PAO base stocks have viscosity indices preferably greater than 130, more preferably greater than 135, still more preferably greater than 140.

Esters in a minor amount may be useful in the lubricating oils of this disclosure. Additive solvency and seal compatibility characteristics may be secured by the use of esters such as the esters of dibasic acids with monoalkanols and the polyol esters of monocarboxylic acids. Esters of the former type include, for example, the esters of dicarboxylic acids such as phthalic acid, succinic acid, sebacic acid, fumaric acid, adipic acid, linoleic acid dimer, malonic acid, alkyl malonic acid, alkenyl malonic acid, etc., with a variety of alcohols such as butyl alcohol, hexyl alcohol, dodecyl alcohol, 2-ethylhexyl alcohol, etc. Specific examples of these types of esters include dibutyl adipate, di(2-ethylhexyl) sebacate, di-n-hexyl fumarate, dioctyl sebacate, diisooctyl azelate, diisodecyl azelate, dioctyl phthalate, didecyl phthalate, dieicosyl sebacate, etc.

Particularly useful synthetic esters are those which are obtained by reacting one or more polyhydric alcohols, preferably the hindered polyols such as the neopentyl polyols; e.g., neopentyl glycol, trimethylol ethane, 2-methyl-2-propyl-1,3-propanediol, trimethylol propane, pentaerythritol and dipentaerythritol with alkanoic acids containing at least 4 carbon atoms, preferably C₅ to C₃₀ acids such as saturated straight chain fatty acids including caprylic acid, capric acids, lauric acid, myristic acid, palmitic acid, stearic acid, arachic acid, and behenic acid, or the corresponding branched chain fatty acids or unsaturated fatty acids such as oleic acid, or mixtures of any of these materials.

Esters should be used in an amount such that the improved wear and corrosion resistance provided by the lubricating oils of this disclosure are not adversely affected.

Non-conventional or unconventional base stocks and/or base oils include one or a mixture of base stock(s) and/or base oil(s) derived from: (1) one or more Gas-to-Liquids (GTL) materials, as well as (2) hydrodewaxed, or hydroisomerized/cat (and/or solvent) dewaxed base stock(s) and/or base oils derived from synthetic wax, natural wax or waxy feeds, mineral and/or non-mineral oil waxy feed stocks such as gas oils, slack waxes (derived from the solvent dewaxing of natural oils, mineral oils or synthetic oils; e.g., Fischer-Tropsch feed stocks), natural waxes, and waxy stocks such as gas oils, waxy fuels hydrocracker bottoms, waxy raffinate, hydrocrackate, thermal crackates, foots oil or other mineral, mineral oil, or even non-petroleum oil derived waxy materials such as waxy materials recovered from coal liquefaction or shale oil, linear or branched hydrocarbyl compounds with carbon number of 20 or greater, preferably 30 or greater and mixtures of such base stocks and/or base oils.

GTL materials are materials that are derived via one or more synthesis, combination, transformation, rearrangement, and/or degradation/deconstructive processes from gaseous carbon-containing compounds, hydrogen-containing compounds and/or elements as feed stocks such as hydrogen, carbon dioxide, carbon monoxide, water, methane, ethane, ethylene, acetylene, propane, propylene, propyne, butane, butylenes, and butynes. GTL base stocks and/or base oils are GTL materials of lubricating viscosity that are generally derived from hydrocarbons; for example, waxy synthesized hydrocarbons, that are themselves derived from simpler gaseous carbon-containing compounds, hydrogen-containing compounds and/or elements as feed stocks. GTL base stock(s) and/or base oil(s) include (1) oils boiling in the lube oil boiling range separated/fractionated from synthesized GTL materials such as, for example, by distillation and subsequently subjected to a final wax processing step which involves either or both of a catalytic dewaxing process, or a solvent dewaxing process, to produce lube oils of reduced/low pour point; (2) synthesized wax isomerates, comprising, for example, hydrodewaxed or hydroisomerized catalytically and/or solvent dewaxed synthesized wax or waxy hydrocarbons; and (3) hydrodewaxed or hydroisomerized catalytically and/or solvent dewaxed Fischer-Tropsch (F-T) material (i.e., hydrocarbons, waxy hydrocarbons, waxes and possible analogous oxygenates); preferably hydrodewaxed or hydroisomerized/followed by catalytically and/or solvent dewaxing dewaxed F-T waxy hydrocarbons, or hydrodewaxed or hydroisomerized/followed by catalytically (or solvent) dewaxing dewaxed, F-T waxes, or mixtures thereof.

GTL base stock(s) and/or base oil(s) derived from GTL materials, especially, hydrodewaxed or hydroisomerized/followed by cat and/or solvent dewaxed wax or waxy feed, preferably F-T material derived base stock(s) and/or base oil(s), are characterized typically as having KV100 of from 2 mm²/s to 50 mm²/s. They are further characterized typically as having pour points of −5° C. to −40° C. or lower (ASTM D97). They are also characterized typically as having viscosity indices of 80 to 140 or greater (ASTM D2270).

In addition, the GTL base stock(s) and/or base oil(s) are typically highly paraffinic (>90% saturates), and may contain mixtures of monocycloparaffins and multicycloparaffins in combination with non-cyclic isoparaffins. The ratio of the naphthenic (i.e., cycloparaffin) content in such combinations varies with the catalyst and temperature used. Further, GTL base stock(s) and/or base oil(s) typically have very low sulfur and nitrogen content, generally containing less than 10 ppm, and more typically less than 5 ppm of each of these elements. The sulfur and nitrogen content of GTL base stock(s) and/or base oil(s) obtained from F-T material, especially F-T wax, is essentially nil. In addition, the absence of phosphorous and aromatics make this materially especially suitable for the formulation of low SAP products.

The term GTL base stock and/or base oil and/or wax isomerate base stock and/or base oil is to be understood as embracing individual fractions of such materials of wide viscosity range as recovered in the production process, mixtures of two or more of such fractions, as well as mixtures of one or two or more low viscosity fractions with one, two or more higher viscosity fractions to produce a blend wherein the blend exhibits a target kinematic viscosity.

The GTL material, from which the GTL base stock(s) and/or base oil(s) is/are derived is preferably an F-T material (i.e., hydrocarbons, waxy hydrocarbons, wax).

Base oils for use in the formulated lubricating oils useful in the present disclosure are any of the variety of oils corresponding to API Group I, Group II, Group III, Group IV, Group V and Group VI oils and mixtures thereof, preferably API Group II, Group III, Group IV, Group V and Group VI oils and mixtures thereof, more preferably the Group III to Group VI base oils due to their exceptional volatility, stability, viscometric and cleanliness features. Minor quantities of Group I stock, such as the amount used to dilute additives for blending into formulated lube oil products, can be tolerated but should be kept to a minimum, i.e. amounts only associated with their use as diluent/carrier oil for additives used on an “as received” basis. Even in regard to the Group II stocks, it is preferred that the Group II stock be in the higher quality range associated with that stock, i.e., a Group II stock having a viscosity index in the range 100<VI<120.

In addition, the GTL base stock(s) and/or base oil(s) are typically highly paraffinic (>90% saturates), and may contain mixtures of monocycloparaffins and multicycloparaffmns in combination with non-cyclic isoparaffins. The ratio of the naphthenic (i.e., cycloparaffin) content in such combinations varies with the catalyst and temperature used. Further, GTL base stock(s) and/or base oil(s) and hydrodewaxed, or hydroisomerized/cat (and/or solvent) dewaxed base stock(s) and/or base oil(s) typically have very low sulfur and nitrogen content, generally containing less than 10 ppm, and more typically less than 5 ppm of each of these elements. The sulfur and nitrogen content of GTL base stock(s) and/or base oil(s) obtained from F-T material, especially F-T wax, is essentially nil. In addition, the absence of phosphorous and aromatics make this material especially suitable for the formulation of low sulfur, sulfated ash, and phosphorus (low SAP) products.

The base stock component of the present lubricating oils will typically be from 50 to 99 wt % of the total composition (all proportions and percentages set out in this specification are by weight unless the contrary is stated) and more usually in the range of 80 to 99 wt %.

### Aromatic Monoester Compound Base Stock and Co-Base Stock Components

The lubricating oil of the present disclosure comprises, as a sole base stock, a primary base stock, or a co-base stock, one or more aromatic monoesters derivable from Guerbet alcohols and aromatic acids, or from Guerbet acids and aromatic alcohols, as described above in connection with the composition in the present disclosure. Unlike alkylated naphthalene Group V base stocks, the base stock and co-base stock components of this disclosure contain oxygen functionality and are more polar. These base stock and co-base stock components exhibit good solvency for polar additives, which make them attractive as Group V synthetic base stocks in lubricant applications.

Aromatic monoester compound base stock and co-base stock components (i.e., a type of composition of the present disclosure) derivable from a branched Guerbet alcohol and an aromatic acid desirably have a KV100 from 3 to 12 cSt, a VI from 25 to 125, and a NV of less than 20%.

Preferred aromatic monoester compound base stock and co-base stock components derivable from a branched Guerbet alcohol and an aromatic acid have a KV100 from 4 to 11, more preferably from 5 to 10, A VI from 30 to 120, more preferably from 35 to 115, even more preferably from 40 to 110, and a Noack volatility of no greater than 18%, more preferably no greater than 15%, even more preferably no greater than 12%.

Aromatic monoester compound base stock and co-base stock components derived from a branched Guerbet acid and an aromatic alcohol have a KV100 from 3 to 12 cSt, a VI from 25 to 125, and a NV of less than 20%.

Preferred aromatic monoester compound base stock and co-base stock components (i.e., a type of composition of the present disclosure) derivable from a branched Guerbet acid and an aromatic alcohol desirably have a KV100 from 4 to 11, more preferably from 5 to 10, a VI from 30 to 120, more preferably from 35 to 115, even more preferably from 40 to 110, and a Noack volatility of no greater than 18%, more preferably no greater than 15%, even more preferably no greater than 12%.

The aromatic monoester compound co-base stock component is preferably present in an amount sufficient for providing desired solvency for polar additives in the lubricating oil. The aromatic monoester compound co-base stock component is present in the lubricating oils of this disclosure in an amount from c1 to c2 wt %, where c1 and c2 can be, independently, 1, 2, 3, 4, 5, 10, 15, 20, 25, 30, 35, 40, 45, 50, as long as c1<c2.

As indicated above, it is also contemplated that the lubricating oil of the present disclosure may contain a mixture of monoesters derivable from Guerbet alcohol and a carboxylic acid and monoesters derivable from Guerbet acid and an alcohol, given their similar structures. The total quantity and desired properties (such as KV100, VI, and NV value) of such mixture should be similar to those mentioned above in connection with using only one type of the monoesters.

### Lubricating Oil Additives

The formulated lubricating oil useful in the present disclosure may additionally contain one or more of the commonly used lubricating oil performance additives including but not limited to dispersants, detergents, viscosity modifiers, antiwear additives, corrosion inhibitors, rust inhibitors, metal deactivators, extreme pressure additives, anti-seizure agents, wax modifiers, viscosity modifiers, fluid-loss additives, seal compatibility agents, lubricity agents, anti-staining agents, chromophoric agents, defoamants, demulsifiers, densifiers, wetting agents, gelling agents, tackiness agents, colorants, and others. For a review of many commonly used additives and the quantities used, see: (i) Klamann in Lubricants and Related Products, Verlag Chemie, Deerfield Beach, Fla.; ISBN 0-89573-177-0; (ii) “Lubricant Additives,” M. W. Ranney, published by Noyes Data Corporation of Parkridge, N J (1973); (iii) “Synthetics, Mineral Oils, and Bio-Based Lubricants,” Edited by L. R. Rudnick, CRC Taylor and Francis, 2006, ISBN 1-57444-723-8; (iv) “Lubrication Fundamentals”, J. G. Wills, Marcel Dekker Inc., (New York, 1980); (v) Synthetic Lubricants and High-Performance Functional Fluids, 2nd Ed., Rudnick and Shubkin, Marcel Dekker Inc., (New York, 1999); and (vi) “Polyalphaolefins,” L. R. Rudnick, Chemical Industries (Boca Raton, Fla., United to States) (2006), 111 (Synthetics, Mineral Oils, and Bio-Based Lubricants), 3-36. Reference is also made to: (a) U.S. Pat. No. 7,704,930 B2; (b) U.S. Pat. No. 9,458,403 B2, Column 18, line 46 to Colum 39, line 68; (c) U.S. Pat. No. 9,422,497 B2, Column 34, line 4 to Colum 40, line 55; and (d) U.S. Pat. No. 8,048,833 B2, Column 17, line 48 to Colum 27, line 12, the disclosures of which are incorporated herein in its entirety. These additives are commonly delivered with varying amounts of diluent oil, that may range from 5 wt % to 50 wt % based on the total weight of the additive package before incorporation into the formulated oil.

The additives useful in this disclosure do not have to be soluble in the lubricating oils. Insoluble additives in oil can be dispersed in the lubricating oils of this disclosure.

The types and quantities of performance additives used in combination with the instant disclosure in lubricant compositions are not limited by the examples shown herein as illustrations.

When lubricating oil compositions contain one or more of the additives discussed above, the additive(s) are blended into the composition in an amount sufficient for it to perform its intended function. Typical amounts of such additives useful in the present disclosure are shown in Table 1 below.

It is noted that many of the additives are shipped from the additive manufacturer as a concentrate, containing one or more additives together, with a certain amount of base oil diluents. Accordingly, the weight amounts in the table below, as well as other amounts mentioned herein, are directed to the amount of active ingredient (that is the non-diluent portion of the ingredient). The weight percent (wt %) indicated below is based on the total weight of the lubricating oil composition.

In the above detailed description, the specific embodiments of this disclosure have been described in connection with its preferred embodiments. However, to the extent that the above description is specific to a particular embodiment or a particular use of this disclosure, this is intended to be illustrative only and merely provides a concise description of the exemplary embodiments. Accordingly, the disclosure is not limited to the specific embodiments described above, but rather, the disclosure includes all alternatives, modifications, and equivalents falling within the true scope of the appended claims. Various modifications and variations of this disclosure will be obvious to a worker skilled in the art and it is to be understood that such modifications and variations are to be included within the purview of this application and the spirit and scope of the claims.

### EXAMPLES

### Example 1

### Synthesis of 2-hexyldecyl 4′-methylbiphenyl-4-carboxylate: Esterification of 2-hexyl-1-decanol with 4′-methylbiphenyl-4-carboxylic acid to Form Monoester Product Catalyzed by p-Toluene Sulfonic Acid in Refluxing Toluene

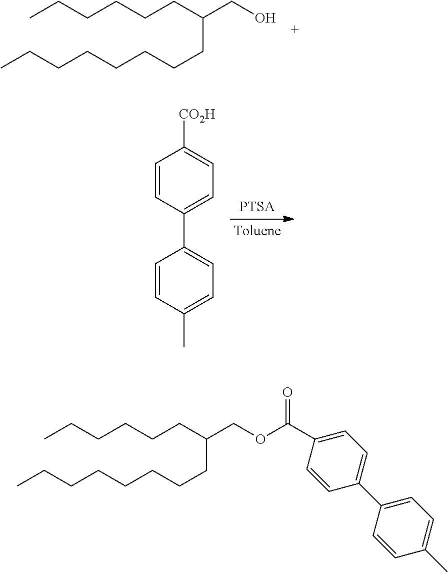 [SMILES: CCCCCCCCC(CO)CCCCCC ; Cc1ccc(-c2ccc(C(=O)O)cc2)cc1 ; CCCCCCCCC(CCCCCC)COC(=O)c1ccc(-c2ccc(C)cc2)cc1]

2-Hexyldecan-1-ol (11.42 g, 0.0471 mol, MW: 242.44), 4′-methylbiphenyl-4-carboxylic acid (5.0 g 0.0236 mol, MW: 212.24) and p-toluenesulfonic acid monohydride (PTSA) (0.449 g, 0.00236 mol, MW: 190.22) were mixed 75 ml toluene in three necked round bottom flask along with a dean-stark apparatus. Then solution was refluxed for overnight (18 h). In 18 hours, 3-4 ml water was collected in the trap. Toluene was removed by simple distillation at 50° C. The product was extracted in hexane (1×100 ml) and washed with saturated NaHCO₃ solution (1×100 ml and water (1×100 ml) until pH ˜7. Separated organic layer dried on anhydrous MgSO₄ and filtered through celite. The hexane layer was removed by rotavapor at 50° C. under vacuum and high boiling components by air bath oven at 180° C. Yields: 7.5 g (72%). The isolated product was characterized by IR and NMR. IR: 3025, 2954, 2926, 2855, 1720, 1608, 1466, 1497, 1417, 1398, 1378, 1309, 1273, 1189, 1177, 1112, 1101, 1019, 1006, 862, 816, 769, 721, 701. 1H NMR (CDCl3): δ 8.09-7.31 (m, 8H, biphenyl ring), 4.25 (d, 2H, O═C—CH2-), 2.40 (S, 3H, —CH3) 1.85 (S, 1H, —CH), 1.42-1.31 (m, 24H, —CH2-), 0.88 (t, 6H, CH3).

### Example 2

### Synthesis of 2-butyloctyl 4′-methylbiphenyl-4-carboxylate: Esterification of 2-butylooctanol with 4′-methylbiphenyl-4-carboxylic acid to Form Monoester Product Catalyzed by p-toluene Sulfonic Acid in Refluxing Toluene

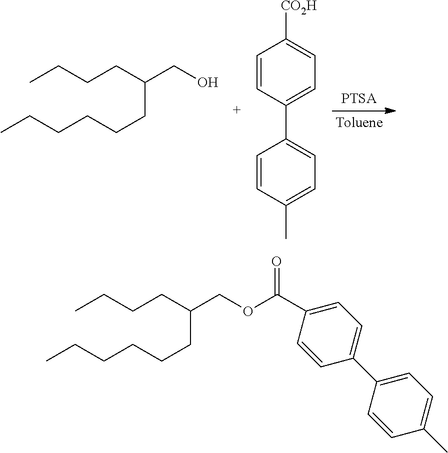

2-Butyloctanol (8.77 g, 0.0471 mol, MW: 186.33), 4′-methylbiphenyl-4-carboxylic acid (5.0 g 0.0236 mol, MW: 212.24) and p-toluenesulfonic acid monohydride (PTSA) (0.449 g, 0.00236 mol, MW: 190.22) were mixed 75 ml toluene in three necked round bottom flask along with a dean-stark apparatus. Then solution was refluxed for overnight (18 h). In 18 hours, 3-4 ml water was collected in the trap. Toluene was removed by simple distillation at 50° C. The product was extracted in hexane (1×100 ml) and washed with saturated NaHCO₃ solution (1×100 ml) and water (2×100 ml) until pH-7. Separated organic layer dried on anhydrous MgSO₄ and filtered through celite. The hexane layer was removed by rotavapor at 50° C. under vacuum and high boiling components by air bath oven at 180° C. Yields: 7.0 g (˜79%). The isolated was product and characterized by IR and NMR. IR: 3025, 2955, 2926, 2857, 1718, 1608, 1527, 1497, 1466, 1417, 1398, 1378, 1309, 1273, 1189, 1117, 1006, 961, 862, 816, 769, 716, 701, 666. 1H NMR (CDCl3): δ 8.09-7.31 (m, 8H, biphenyl ring), 4.25 (d, 2H, O═C—CH2-), 2.42 (S, 3H, —CH3) 1.82-1.34 (m, 17H, —CH2-), 0.89 (t, 6H, CH3).

### Example 3

### Lube Properties

The lube properties of the products of Examples 1 and 2 were evaluated and the data are shown below. The results are shown in TABLE I below. In this table, the AN5 base stock is a alkyl naphthalene type Group V base stock commercially available from ExxonMobil Chemical Company having an address at 5200 Bayway Drive, Baytown, Tex., United States.

TABLE I

| | KV100 | KV40 | | |
|---|---|---|---|---|
| Fluid | (cSt) | (cSt) | VI | NV (%) |
| Example 1 | 6.4 | 15.6 | 84 | 5.6 (Thermogravimetric Analysis) |
| Example 2 | 5.2 | 36.9 | 49 | 14.0 (Thermogravimetric Analysis) |
| Alkylated | 4.7 | 29.0 | 74 | 12.7 |
| Naphthalene | | | | |
| (AN5) | | | | |

The above table shows that the fluids prepared in Examples 1 and 2 above have properties desirable for certain base stocks for lubricants. The data also show that by changing Guerbet type alcohol such as higher alcohol (C20-alcohol), lower alcohol (C8 alcohol), or mixed alcohol and aromatic acids such as naphthoic acid, lube properties can be varied. Similarly, a Guerbet acid can be used to react with various alcohols to make ester fluids with similar molecular structures. These fluids may combine the properties of two leading Group V fluids, i.e., alkyl naphthalenes and esters, used in the industry today.

All patents and patent applications, test procedures (such as ASTM methods, UL methods, and the like), and other documents cited herein are fully incorporated by reference to the extent such disclosure is not inconsistent with this disclosure and for all jurisdictions in which such incorporation is permitted.

When numerical lower limits and numerical upper limits are listed herein, ranges from any lower limit to any upper limit are contemplated. While the illustrative embodiments of the disclosure have been described with particularity, it will be understood that various other modifications will be apparent to and can be readily made by those skilled in the art without departing from the spirit and scope of the disclosure. Accordingly, it is not intended that the scope of the claims appended hereto be limited to the examples and descriptions set forth herein but rather that the claims be construed as encompassing all the features of patentable novelty which reside in the present disclosure, including all features which would be treated as equivalents thereof by those skilled in the art to which the disclosure pertains.

The present disclosure has been described above with reference to numerous embodiments and specific examples. Many variations will suggest themselves to those skilled in this art in light of the above detailed description. All such obvious variations are within the full intended scope of the appended claims.

What is claimed is:

1. A composition comprising (i) a first compound having a formula (F-I) below and/or (ii) a second compound having a formula (F-II) below: 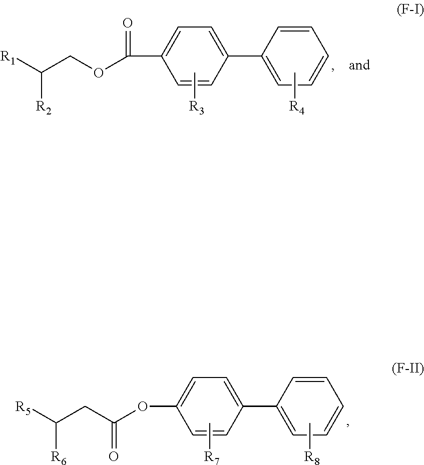
wherein R₁, R₂, R₅, and R₆ are independently a substituted or unsubstituted alkyl group having from 2 to 30 carbon atoms, and R₃, R₄, R₇, and R₈ are independently hydrogen or a substituted or unsubstituted alkyl group having from 1 to 8 carbon atoms.

2. The composition of claim 1, wherein the first compound comprises at least one of: 2-hexyldecyl 4′-methylbiphenyl-4-carboxylate, 2-butyloctyl 4′-methylbiphenyl-4-carboxylate, 2-ethylhexyl 4′-methyl-[1,1′-biphenyl]-4-carboxylate, 2-octyldodecyl 4′-methyl-[1,1′-biphenyl]-4-carboxylate, 2-decyltetradecyl 4′-methyl-[1,1′-biphenyl]-4-carboxylate, 2-butyloctyl [1,1′-biphenyl]-4-carboxylate, 2-ethylhexyl [1,1′-biphenyl]-4-carboxylate, 2-hexyldecyl [1,1′-biphenyl]-4-carboxylate, 2-octyldodecyl [1,1′-biphenyl]-4-carboxylate, and 2-decyltetradecyl [1,1′-biphenyl]-4-carboxylate.

3. The composition of claim 1, wherein the second compound comprises at least one of: [1,1′-biphenyl]-4-yl 2-decyltetradecanoate, [1,1′-biphenyl]-4-yl 2-octyldodecanoate, [1,1′-biphenyl]-4-yl 2-hexyldecanoate, [1,1′-biphenyl]-4-yl 2-butyloctanoate, [1,1′-biphenyl]-4-yl 2-ethylhexanoate, [1,1′-biphenyl]-3-yl 2-decyltetradecanoate, [1,1′-biphenyl]-3-yl 2-octyldodecanoate, [1,1′-biphenyl]-3-yl 2-hexyldecanoate, [1,1′-biphenyl]-3-yl 2-butyloctanoate, [1,1′-biphenyl]-3-yl 2-ethylhexanoate, [1,1′-biphenyl]-2-yl 2-decyltetradecanoate, [1,1′-biphenyl]-2-yl 2-octyldodecanoate, [1,1′-biphenyl]-2-yl 2-hexyldecanoate, [1,1′-biphenyl]-2-yl 2-butyloctanoate, [1,1′-biphenyl]-2-yl 2-ethylhexanoate, 3′-methoxy-[1,1′-biphenyl]-2-yl 2-decyltetradecanoate, 3′-methoxy-[1,1′-biphenyl]-2-yl 2-octyldodecanoate, 3′-methoxy-[1,1′-biphenyl]-2-yl 2-hexyldecanoate, 3′-methoxy-[1,1′-biphenyl]-2-yl 2-butyloctanoate, 3′-methoxy-[1,1′-biphenyl]-2-yl 2-ethylhexanoate, 3-amino-[1,1′-biphenyl]-4-yl 2-decyltetradecanoate, 3-amino-[1,1′-biphenyl]-4-yl 2-octyldodecanoate, 3-amino-[1,1′-biphenyl]-4-yl 2-hexyldecanoate, 3-amino-[1,1′-biphenyl]-4-yl 2-butyloctanoate, 3-amino-[1,1′-biphenyl]-4-yl 2-ethylhexanoate, 3′-formyl-[1,1′-biphenyl]-4-yl 2-decyltetradecanoate, 3′-formyl-[1,1′-biphenyl]-4-yl 2-octyldodecanoate, 3′-formyl-[1,1′-biphenyl]-4-yl 1 2-hexyldecanoate, 3′-formyl-[1,1′-biphenyl]-4-yl 2-butyloctanoate, and 3′-formyl-[1,1′-biphenyl]-4-yl 2-ethylhexanoate.

4. The composition of claim 1, wherein the composition has a kinematic viscosity at 100° C. as determined by ASTM D445 (“KV100”) from 3 to 12 cSt, a viscosity index as determined by ASTM D2270 (“VI”) of from 25 to 125, and a Noack volatility as determined by ASTM D5800 (“NV”) of less than 20%.

5. The composition of claim 1, which is a lubricant base stock.

6. A lubricating oil comprising a composition of claim 1.

7. A lubricating oil comprising of claim 6, comprising at least one of:
2-hexyldecyl 4′-methylbiphenyl-4-carboxylate, 2-butyloctyl 4′-methylbiphenyl-4-carboxylate, 2-ethylhexyl 4′-methyl-[1,1′-biphenyl]-4-carboxylate, 2-octyldodecyl 4′-methyl-20 [1,1′-biphenyl]-4-carboxylate, 2-decyltetradecyl 4′-methyl-[1,1′-biphenyl]-4-carboxylate, 2-butyloctyl [1,1′-biphenyl]-4-carboxylate, 2-ethylhexyl [1,1′-biphenyl]-4-carboxylate, 2-hexyldecyl [1,1′-biphenyl]-4-carboxylate, 2-octyldodecyl [1,1′-biphenyl]-4-carboxylate, 2-decyltetradecyl [1,1′-biphenyl]-4-carboxylate;
[1,1′-biphenyl]-4-yl 2-decyltetradecanoate, [1,1′-biphenyl]-4-yl 2-octyldodecanoate, [1,1′-biphenyl]-4-yl 2-hexyldecanoate, [1,1′-biphenyl]-4-yl 2-butyloctanoate, [1,1′-biphenyl]-4-yl 2-ethylhexanoate, [1,1′-biphenyl]-3-yl 2-decyltetradecanoate, [1,1′-biphenyl]-3-yl 2-octyldodecanoate, [1,1′-biphenyl]-3-yl 2-hexyldecanoate, [1,1′-biphenyl]-3-yl 2-butyloctanoate, [1,1′-biphenyl]-3-yl 2-ethylhexanoate, [1,1′-biphenyl]-2-yl 2-decyltetradecanoate, [1,1′-biphenyl]-2-yl 2-octyldodecanoate, [1,1′-biphenyl]-2-yl 2-hexyldecanoate, [1,1′-biphenyl]-2-yl 2-butyloctanoate, [1,1′-biphenyl]-2-yl 2-ethylhexanoate, 3′-methoxy-[1,1′-biphenyl]-2-yl 2-decyltetradecanoate, 3′-methoxy-[1,1′-biphenyl]-2-yl 2-octyldodecanoate, 3′-methoxy-[1,1′-biphenyl]-2-yl 2-hexyldecanoate, 3′-methoxy-[1,1′-biphenyl]-2-yl 2-butyloctanoate, 3′-methoxy-[1,1′-biphenyl]-2-yl 2-ethylhexanoate, 3-amino-[1,1′-biphenyl]-4-yl 2-decyltetradecanoate, 3-amino-[1,1′-biphenyl]-4-yl 2-octyldodecanoate, 3-amino-[1,1′-biphenyl]-4-yl 2-hexyldecanoate, 3-amino-[1,1′-biphenyl]-4-yl 2-butyloctanoate, 3-amino-[1,1′-biphenyl]-4-yl 2-ethylhexanoate, 3′-formyl-[1,1′-biphenyl]-4-yl 2-decyltetradecanoate, 3′-formyl-[1,1′-biphenyl]-4-yl 2-octyldodecanoate, 3′-formyl-[1,1′-biphenyl]-4-yl 1 2-hexyldecanoate, 3′-formyl-[1,1′-biphenyl]-4-yl 2-butyloctanoate, and 3′-formyl-[1,1′-biphenyl]-4-yl 2-ethylhexanoate.

8. The lubricating oil of claim 6, wherein the lubricating oil has a KV 100 from 3 to 12 cSt, a VI from 25 to 125, and a NV less than 20%.

9. The lubricating oil of claim 6, wherein the composition is present in an amount from 1 to 20 wt %, based on the total weight of the lubricating oil.

10. The lubricating oil of claim 6, wherein the lubricating oil further comprises:
(i) at least one major base stock differing from the composition, in an amount in the range from 20 to 80 wt %, based on the total weight of the lubricating oil; and
(ii) a lubricant additive.

11. A process for making a composition, comprising a step (A) or a step (B), or both steps (A) and (B), as follows:
(A) reacting a Guerbet alcohol with an aromatic acid optionally in the presence of a first catalyst and a first solvent, under reaction conditions sufficient to produce a first compound which comprises a Guerbet alcohol-based aromatic ester; wherein the Guerbet alcohol comprises 8 to 32 carbon atoms, and the aromatic acid is an aromatic mono-carboxylic acid having from 8 to 32 carbon atoms; and
(B) reacting an aromatic alcohol with a Guerbet acid, optionally in the presence of a second catalyst and a second solvent, under reaction conditions sufficient to produce a second compound which comprises a Guerbet acid-based aromatic ester; wherein the Guerbet acid comprises 8 to 32 carbon atoms, and the aromatic alcohol is an aromatic mono-alcohol having from 8 to 32 carbon atoms.

12. The process of claim 11, further comprising:
(C) mixing one or more Guerbet alcohol-based aromatic esters derived from step (A) with one or more Guerbet acid-based aromatic esters derived from step (B).

13. The process of claim 11, wherein in step (A):
the Guerbet alcohol comprises at least one of: 2-hexyl-1-decanol, 2-butyloctanol, 2-hexyl-1-octanol, 2-hexyl-1-decanol, 2-octyl-1-decanol, 2-octyl-1-dodecanol, 2-decyl-1-dodecanol, 2-decyl-1-tetradecanol, 2-heptyl-l-undecanol, 2-ethyl-1-hexanol, 2-butyl-1-hexanol, and 2-butyl-1-octanol; and
the aromatic acid comprises at least one of: 4′-methylbiphenyl-4-carboxylic acid, biphenyl-2-carboxylic acid, biphenyl-4-carboxylic acid, biphenyl-3-carboxylic acid, 4′-(trifluoromethyl)-2-biphenylcarboxylic acid, 4′-hydroxy-4-biphenylcarboxylic acid, 4′-methyl-4-biphenylcarboxylic acid, 4′-formyl-4-biphenylcarboxylic acid, 4′-bromo-4-biphenylcarboxylic acid, 3′-bromo-3-biphenylcarboxylic acid, 3′-(fluorophenyl)benzoic acid, 4′-amino-4-biphenylcarboxylic acid, 3′-amino-3-biphenylcarboxylic acid, 2-biphenyl-[1,3]dioxol-5-yl-carboxylic acid, 3-biphenyl-[1,3]dioxol-5-yl-carboxylic acid, 2-biphenyl-(2′-methoxy)acetic acid, 4-biphenyl[l1,3]dioxo-5-yl-acetic acid, 4-biphenyl-3′-amino acetic acid, 5-amino-biphenyl-2-carboxylic acid, (2-methoxy-biphenyl-4-yl)acetic acid, 2-cyano-biphenyl-2-carboxylic acid, 4-cyano-biphenyl-2-carboxylic acid, 4-formyl-biphenyl-4-carboxylic acid, biphenyl-4-carboxylic acid, biphenyl-3-carboxylic acid, 1-naphthalenecarboxylic acid, 2-naphthalenecarboxylic acid, 4-methyl-l-napthoic acid, 1-napthoic acid, and 2-napthoic acid.

14. The process of claim 11, wherein in step (B):
the Guerbet acid comprises at least one of: 2-ethylhexanoic acid, 2-butylhexanoic acid, 2-butyloctanoic acid, 2-hexyldecanoic acid, 2-heptylundecanoic acid, 2-octyldecanoic acid, 2-decyldodecanoic acid, and isotridecanoic acid; and
the aromatic alcohol comprises at least one of: 1-naphthol, 2-naphthol, 2-methyl-1-naphthol, 3-methoxy-2-naphthol, 3-methoxynaphthalen-1-ol, 4-methoxy-1-naphthol, 6-methoxy-2-naphthol, 2-phenylphenol, 3-phenylphenol, 4-phenylphenol, 4-phenoxyphenol, 2-amino-4-phenylphenol, 3′-hydroxy-biphenyl-3-carbaldehyde, 4′-hydroxy-biphenyl-3-carbaldehyde, and 3-methoxy[1,1′-biphenyl]-3-ol.

15. The process of claim 11, wherein the first compound comprises at least one of: 2-hexyldecyl 4′-methylbiphenyl-4-carboxylate, 2-butyloctyl 4′-methylbiphenyl-4-carboxylate, 2-ethylhexyl 4′-methyl-[1,1′-biphenyl]-4-carboxylate, 2-octyldodecyl 4′-methyl-[1,1′-biphenyl]-4-carboxylate, 2-decyltetradecyl 4′-methyl-[1,1′-biphenyl]-4-carboxylate, 2-butyloctyl [1,1′-biphenyl]-4-carboxylate, 2-ethylhexyl [1,1′-biphenyl]-4-carboxylate, 2-hexyldecyl [1,1′-biphenyl]-4-carboxylate, 2-octyldodecyl [1,1′-biphenyl]-4-carboxylate, and 2-decyltetradecyl [1,1′-biphenyl]-4-carboxylate.

16. The process of claim 11, wherein the second compound comprises at least one of: [1,1′-biphenyl]-4-yl 2-decyltetradecanoate, [1,1′-biphenyl]-4-yl 2-octyldodecanoate, [1,1′-biphenyl]-4-yl 2-hexyldecanoate, [1,1′-biphenyl]-4-yl 2-butyloctanoate, [1,1′-biphenyl]-4-yl 2-ethylhexanoate, [1,1′-biphenyl]-3-yl 2-decyltetradecanoate, [1,1′-biphenyl]-3-yl 2-octyldodecanoate, [1,1′-biphenyl]-3-yl 2-hexyldecanoate, [1,1′-biphenyl]-3-yl 2-butyloctanoate, [1,1′-biphenyl]-3-yl 2-ethylhexanoate, [1,1′-biphenyl]-2-yl 2-decyltetradecanoate, [1,1′-biphenyl]-2-yl 2-octyldodecanoate, [1,1′-biphenyl]-2-yl 2-hexyldecanoate, [1,1′-biphenyl]-2-yl 2-butyloctanoate, [1,1′-biphenyl]-2-yl 2-ethylhexanoate, 3′-methoxy-[1,1′-biphenyl]-2-yl 2-decyltetradecanoate, 3′-methoxy-[1,1′-biphenyl]-2-yl 2-octyldodecanoate, 3′-methoxy-[1,1′-biphenyl]-2-yl 2-hexyldecanoate, 3′-methoxy-[1,1′-biphenyl]-2-yl 2-butyloctanoate, 3′-methoxy-[1,1′-biphenyl]-2-yl 2-ethylhexanoate, 3-amino-[1,1′-biphenyl]-4-yl 2-decyltetradecanoate, 3-amino-[1,1′-biphenyl]-4-yl 2-octyldodecanoate, 3-amino-[1,1′-biphenyl]-4-yl 2-hexyldecanoate, 3-amino-[1,1′-biphenyl]-4-yl 2-butyloctanoate, 3-amino-[1,1′-biphenyl]-4-yl 2-ethylhexanoate, 3′-formyl-[1,1′-biphenyl]-4-yl 2-decyltetradecanoate, 3′-formyl-[1,1′-biphenyl]-4-yl 2-octyldodecanoate, 3′-formyl-[1,1′-biphenyl]-4-yl 1 2-hexyldecanoate, 3′-formyl-[1,1′-biphenyl]-4-yl 2-butyloctanoate, and 3′-formyl-[1,1′-biphenyl]-4-yl 2-ethylhexanoate.

17. The process of claim 11, wherein at least one of (i) the first compound, (ii) the second compound, and (iii) mixtures of the first compound and the second compound has a kinematic viscosity at 100° C. as determined by ASTM D445 (KV100) from 3 to 12 cSt, a viscosity index as determined by ASTM D2270 (VI) of from 25 to 125, and a Noack volatility as determined by ASTM D5800 of less than 20%.

18. The process claim 11, wherein the first catalyst and second catalyst independently comprises p-toluenesulfonic acid monohydride (PTSA), titanium (VI) isopropoxide or sulfuric acid.

19. The process of claim 11, wherein the first solvent and the second solvent independently comprises toluene, a xylene, a pentane isomer, a hexane isomer, a heptane isomer, or mixtures thereof.

20. A composition obtainable by a process of claim 11.

21. The composition of claim 20, which is a lubricating oil base stock.

22. A lubricating oil comprising a lubricating oil base stock of claim 21.
